(19) United States
(12) Patent Application Publication
(10) Pub. No.: US 2018/0317895 A1
(43) Pub. Date: Nov. 8, 2018

(54) ENDOSCOPIC BIOPSY NEEDLE TIP AND METHODS OF USE

(71) Applicant: HOYA Corporation, Tokyo (JP)

(72) Inventors: Christopher Kadamus, West Roxbury, MA (US); Robb Morse Gavalis, Westborough, MA (US); Jason Zachary Moore, State College, PA (US)

(73) Assignee: HOYA Corporation, Tokyo (JP)

(21) Appl. No.: 15/587,775

(22) Filed: May 5, 2017

(51) Int. Cl. A61B 10/04; A61B 10/02

(52) U.S. Cl. CPC A61B 10/04; A61B 2010/045; A61B 10/0266

(57) ABSTRACT

Embodiments of the present disclosure are directed to systems and methods for acquiring a tissue sample in an endoscopic procedure. In one implementation, a biopsy needle is provided. The biopsy needle includes an elongated body extending along a longitudinal axis. The elongated body includes a lumen extending therethrough and a distal end. The distal end includes at least two tines and a plurality of cutouts. Each of the tines includes two ground bevels formed on two grind planes. Each cutout resides between two adjacent tines and includes a V-shaped section. Each cutout may further include a longitudinally straight section.

### BACKGROUND

### Technical Field

The present disclosure generally relates to endoscopic systems and methods of use. More particularly, and without limitation, the disclosed embodiments relate to devices, systems, and methods for endoscopic tissue collection.

### Background Description

Fine needle biopsy (FNB) and fine needle aspiration (FNA) are commonly employed during Endoscopic Ultrasound (EUS) procedures to acquire tissue samples that would normally be collected through open surgical or percutaneous techniques in the past. For example, endoscopic ultrasound-guided fine needle aspiration (EUS-FNA) or ultrasound-guided fine needle biopsy (EUS-FNB) has become an effective and minimally invasive diagnostic sampling method in patients with gastrointestinal or pancreatic lesions. EUS-FNA and EUS-FNB combine endoscopic visualization with ultrasound imaging and a sampling device. This allows physicians to use traditional endoscopic visualization to guide their way through the gastrointestinal tract and use ultrasound imaging to provide images of organs and structures beyond the wall of the tract to guide sampling of a desired location. Then, an elongated biopsy needle device is passed through the biopsy channel of the endoscope and is visualized ultrasonically as it penetrates to the desired sampling location to collect a tissue or biological liquid sample.

One of the main considerations to optimize EUS-FNA or EUS-FNB procedures is to acquire an adequate tissue sample with minimal number of passes and a minimal risk of injury to surrounding tissue in the patient. Despite the widespread usage of EUS-FNA and EUS-FNB techniques, one limitation of these techniques is that they often provide tissue samples with scant cellularity and/or lack of histologic architecture. This inadequacy of the collected tissue samples limits the performance of histologic grading of malignant tissues and/or subsequent molecular biological analysis. Another limitation of these techniques is the unclear number of passes or times a needle device must be inserted into the endoscope as needed to acquire adequate tissue samples.

Existing endoscopic needles are not optimized to overcome the limitations of EUS-FNA or EUS-FNB procedures discussed above. For example, the majority of existing endoscopic FNA or FNB needles have a needle tip geometry similar to that of a traditional lancet needle. Lancet needles were designed in the early twentieth century and have been commonly used for injection or percutaneous puncture. Thus, lancet needles are designed to cut and split tissue with the lowest penetration force, apply the least drag to reduce pain, and prevent tissue from entering the needle inner lumen. This is contrary to the tissue collection purpose of an endoscopic FNA or FNB needle. The use of lancet style needle tip geometry in endoscopic FNA or FNB needles results in highly variable success rates of collecting tissue samples.

Other endoscopic FNA or FNB needles use alternative needle tip geometries, such as back bevel needles, Franseen needles, and offset tine needles. However, the acquisition rate of adequate amount of tissue could still be improved. This is primarily due to the small gauge of endoscopic FNA or FNB needles, the difficulty in getting an endoscopic needle to the correct sampling location, and the needle tip geometry. Depending on the reporting physician, adequate tissue samples can be collected at a success rate as low as 50% using these needles. Variation in patient population and skill of the physician can further vary the success rate considerably. Failure to acquire adequate tissue samples may result in longer anesthetization of the patient, the use of more devices and/or more passes of a device, and even additional procedures to obtain adequate tissue samples for some patients,

Therefore, there is a need for improved biopsy needles or biopsy needle tips that increase the effectiveness of tissue sample collection in EUS-FNA or EUS-FNB procedures, and thus reduce the passes performed by the physician to acquire an adequate tissue sample. Such biopsy needles or biopsy needle tips thus improve the efficiency and success rates of EUS-FNA or EUS-FNB procedures.

### SUMMARY

The embodiments of the present disclosure include devices, systems, and methods for acquiring a tissue sample in an endoscopic procedure. Advantageously, the exemplary embodiments may allow for the acquisition of adequate tissue or biological liquid samples in EUS-FNA or EUS-FNB procedures with high success rates, thereby improving the efficiency and effectiveness of these endoscopic procedures.

According to an exemplary embodiment of the present disclosure, a biopsy needle is described. The biopsy needle includes an elongated body extending along a longitudinal axis. The elongated body includes a lumen extending therethrough and a distal end. The distal end includes at least four tines and a plurality of cutouts. Each of the tines includes two ground bevels formed on two grind planes. Each cutout resides between two adjacent tines and includes a V-shaped section. Each cutout may further include a longitudinally straight section.

According to a further exemplary embodiment of the present disclosure, a device for needle biopsy is described. The device includes a biopsy needle having an elongated body extending along a longitudinal axis and a distal end. The distal end includes at least three tines and a plurality of cutouts. Each of the tines includes two ground bevels formed on two grind planes. Each cutout resides between two adjacent tines and includes a V-shaped section. Each cutout may further include a longitudinally straight section.

According to a yet further exemplary embodiment of the present disclosure, a biopsy needle tip. The biopsy needle tip includes at least two tines and a plurality of cutouts. Each of the tines includes two ground bevels formed on two grind planes. Each cutout resides between two adjacent tines and includes a V-shaped section. Each cutout may further include a longitudinally straight section. The lengths of the cutouts along the longitudinal axis are greater than the widths of the tines.

Additional features and advantages of the disclosed embodiments will be set forth in part in the description that follows, and in part will be obvious from the description, or may be learned by practice of the disclosed embodiments. The features and advantages of the disclosed embodiments will be realized and attained by the elements and combinations particularly pointed out in the appended claims.

It is to be understood that both the foregoing general description and the following detailed description are examples and explanatory only and are not restrictive of the disclosed embodiments as claimed.

The accompanying drawings constitute a part of this specification. The drawings illustrate several embodiments of the present disclosure and, together with the description, serve to explain the principles of the disclosed embodiments as set forth in the accompanying claims.

### BRIEF DESCRIPTION OF THE DRAWINGS

FIG. 1 is a partial perspective view of an exemplary biopsy needle, according to embodiments of the present disclosure.

FIG. 2 is another partial perspective view of the exemplary biopsy needle of FIG. 1, according to embodiments of the present disclosure.

FIG. 3A is a partial front view of another exemplary biopsy needle without cutouts.

FIG. 3B is a partial front view of the exemplary biopsy needle of FIG. 1, according to embodiments of the present disclosure.

FIG. 3C is a partial front view of another exemplary biopsy needle, according to embodiments of the present disclosure.

FIG. 3D is a partial front view of another exemplary biopsy needle, according to embodiments of the present disclosure.

FIG. 4 is a partial perspective view of another exemplary biopsy needle, according to embodiments of the present disclosure.

FIG. 5 is a partial perspective view of another exemplary biopsy needle, according to embodiments of the present disclosure.

FIG. 6 is a graphical illustration for an exemplary distribution of cutting force along the distal end of the exemplary biopsy needle of FIG. 4.

FIG. 7 is a graphical illustration for an exemplary distribution of cutting force along the distal end of the exemplary biopsy needle of FIG. 5.

FIG. 8 is a graphical illustration for an exemplary distribution of cutting force along the distal end of the exemplary biopsy needle of FIG. 1.

FIG. 9 is a partial front view of another exemplary biopsy needle, according to embodiments of the present disclosure.

FIG. 10 is a partial perspective view of the exemplary biopsy needle of FIG. 9, according to embodiments of the present disclosure.

FIG. 11 is a partial perspective view of the exemplary biopsy needle of FIG. 9 during insertion, according to embodiments of the present disclosure.

FIG. 12 is a partial perspective view of the exemplary biopsy needle of FIG. 9 during extraction, according to embodiments of the present disclosure.

### DETAILED DESCRIPTION

The disclosed embodiments relate to devices, systems, and methods for collecting an adequate tissue sample in an endoscopic procedure. Embodiments of the present disclosure can be implemented in an endoscopic system for collecting tissue samples at desired locations in or in proximity of the gastrointestinal or pancreatic tract, where soft tissue samples are typically collected for diagnostic biopsy. Advantageously, embodiments of the present disclosure allow for effective collection of a desired amount of tissue sample at a desired location, thereby increasing the success rate and efficiency of collecting adequate tissue samples in an endoscopic procedure.

As described herein, an endoscope, such as an ultrasound endoscope, typically includes a proximal end, a distal end, and an internal working channel extending between the distal end and the proximal end. A proximal end may refer to a point or a location along the length of the endoscope closer to a physician or a medical practitioner. A distal end may refer to a point or location along the length of the endoscope closer to a sampling location in the body of a patient. A biopsy needle device is typically introduced into the working channel of the endoscope from the proximal end to the distal end of the endoscope until a distal end of the needle device approximates or reaches a desired location for collecting one or more tissue samples.

According to an aspect of the present disclosure, a biopsy needle for collecting a tissue sample is described. The biopsy needle includes an elongated body extending along a longitudinal axis. The elongated body includes a lumen extending therethrough and a distal end or a needle tip having a plurality of tines. The tines may each have a symmetric shape. Also, the tines may each have two ground bevels formed on two grind planes that meet at a tine tip. Each of the two grind planes is oblique relative to the longitudinal axis at a desired bevel angle. In some embodiments, the two grind planes may be oblique relative to the longitudinal axis at a same desired bevel angle. The bevel angle may be predetermined based on a selection of factors, including the hardness of the tissue, the number of tines, the geometry of the tines, etc.

According to an aspect of the present disclosure, the tines of the biopsy needle are evenly radially spaced apart in rotational symmetry. The tines may be further arranged in plane symmetry about two longitudinal planes that are orthogonal to each other and parallel to the longitudinal axis of the biopsy needle. The rotational symmetric and/or plane symmetric arrangement of the tines of the biopsy needle allows substantially balanced cutting force to be applied to the tissue being cut by the distal end of the biopsy needle during needle insertion. The substantially balanced cutting force allows the tissue being cut, which would be pushed away from or pushed around the needle by unbalanced forces, to move towards the center of the biopsy needle and to enter the lumen of the biopsy needle. Thus, the rotational symmetric and/or plane symmetric arrangement of the tines advantageously increases the amount and/or length of the collected tissue sample, thereby increasing the success rate of collecting an adequate tissue sample.

Additionally, the substantially balanced cutting force applied to the tissue being cut by the distal end of the biopsy needle in turn results in substantially balanced reactive force of the tissue applied to the distal end. The substantially balanced reactive force prevents the biopsy needle from being veered off course, changing direction, and/or curving in undesired directions. Thus, the rotational symmetric and/or plane symmetric arrangement of the tines advantageously allows the biopsy needle to maintain integrity and/or a straight sampling path during needle insertion, thereby increasing the accuracy of sampling a desired legion location.

According to another aspect of the present disclosure, a biopsy needle including four tines at its distal end is described. The four tines are evenly radially spaced apart in rotational symmetry and in plane symmetry about two orthogonal longitudinal planes that are parallel to the longitudinal axis. The tines are formed on four grind planes. Each of the four grind planes is oblique to the longitudinal axis at the same bevel angle as well as orthogonal to two neighboring grind planes in an x-y plane perpendicular to the longitudinal axis. Advantageously, compared to a needle with two tines and three tines, a biopsy needle with four tines allows forces applied to the distal end of the needle to remain substantially balanced during the insertion of the needle and cutting of the tissue. Additionally, when extracted from the tissue after insertion, the exemplary biopsy needle with four tines reduces the loss of tissue by providing more supporting and/or frictional surfaces surrounding the cut tissue sample, thereby increasing the success rate of collecting an adequate tissue sample.

According to another aspect of the present disclosure, the biopsy needle further includes a plurality of cutouts at its distal end. The cutouts each reside between two adjacent tines. For example, a biopsy needle with four tines may have four primary ground bevels formed on four grind planes. The four cutouts may cut into or cut through the heels of the four primary ground bevels respectively, eliminating the heels and separating each of the primary ground bevels into two secondary ground bevels on the same grind plane. The two secondary ground bevels each have a cutting edge. Advantageously, the replacement of the heels of the primary ground bevels with the cutouts eliminates the poor cutting condition at the heels and reduces the overall cutting forces of the biopsy needle, thereby allowing for more efficient tissue cutting and more effective collection of adequate tissue samples.

The cutouts may each include a V-shaped section and/or a longitudinally straight section. In some embodiments, each cutout cuts into or resides within a heel of a primary ground bevel of a biopsy needle. In such instances, the V-shaped section and/or the longitudinally straight section may reside within a lowest point of a grind plane of the primary ground bevel along the longitudinal axis of the biopsy needle. In other embodiments, each cutout cuts through or extends beyond the heel of the primary ground bevel of the biopsy needle. In such instances, the V-shaped section and/or the longitudinally straight section may extend beyond a lowest point of a grind plane of the primary ground bevel along the longitudinal axis of the biopsy needle. The dimensions and/or shapes of the V-shaped section and/or the longitudinally straight section of the cutouts of the biopsy needle may be predetermined such that the integrity of the tines is maintained during insertion and/or extraction of the biopsy needle and such that the heels of the primary ground bevels are eliminated.

In some embodiments, the lengths of the cutouts along the longitudinal axis are greater than the widths of the tines. Cutouts longer than the widths of the tines may allow the tines to be radially deflectable. For example, as the biopsy needle is inserted into a sampling location, the tines may radially deflect outward relatively to the longitudinal axis. As the biopsy needle is extracted from the sampling location, the tines may radially collapse inward relatively to the longitudinal axis. Advantageously, the radial deflection of the tines allows the tissue cut by the distal end to enter the lumen of the biopsy needle more easily (e.g., with less hindrance) during needle insertion and to retain the tissue within the lumen of the biopsy needle (e.g., with more frictional and/or supporting surface) during needle extraction.

Reference will now be made in detail to embodiments and aspects of the present disclosure, examples of which are illustrated in the accompanying drawings. Where possible, the same reference numbers will be used throughout the drawings to refer to the same or like parts.

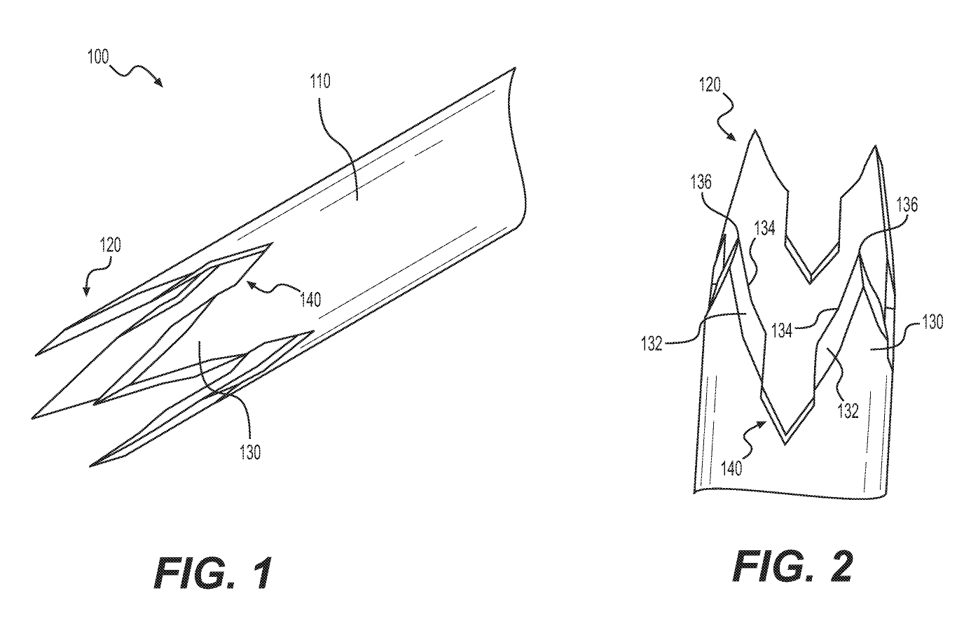

FIGS. 1 and 2 are partial perspective views of an exemplary biopsy needle 100. As shown in FIGS. 1 and 2, biopsy needle 100 includes an elongated body 110 extending along a longitudinal axis. Elongated body 110 includes a distal end 120 and a lumen extending therethrough. As described herein, distal end 120 may also be referred to as the biopsy needle tip. Distal end 120 has a plurality of tines 130 and a plurality of cutouts 140. Tines 130 may each have a symmetric shape and formed by two grind planes. For example, as shown in FIG. 2, tines 130 may each have two ground bevels 132 formed on two grind planes that meet at a tine tip 136. Each ground bevel 132 has a cutting edge 134. Every point along cutting edge 134 of ground bevel 132 has an inclination angle.

As described herein, the inclination angle is the angle between the tangent at a given point of a cutting edge of a needle and the plane perpendicular to the direction of needle insertion. An overall average inclination angle of a needle is an average inclination angle of all the points of all the cutting edges of the needle. For example, as shown in FIG. 2, the inclination angle of cutting edge 134 is at its highest value at tine tip 136 and decreases towards the points at lower portions of ground bevel 132. An overall average inclination angle of biopsy needle 100 is the average inclination angle of all points or locations of all the cutting edges 134 of distal end 120.

As described herein, the inclination angle affects the cutting force applied to the tissue at a given point of a cutting edge. Increasing the inclination angel of a cutting edge of a biopsy needle reduces the cutting forces applied to the tissue being cut, which in turn leads to more efficient tissue cutting and better tissue samples for biopsy (e.g., longer tissue sample or tissue sample with sufficient cellularity and/or histologic architecture). Advantageously, to increase the inclination angle of cutting edge 134 and/or the overall average inclination angle of biopsy needle 100, as shown in FIGS. 1 and 2, distal end 120 of biopsy needle 100 further includes a plurality of cutouts 140.

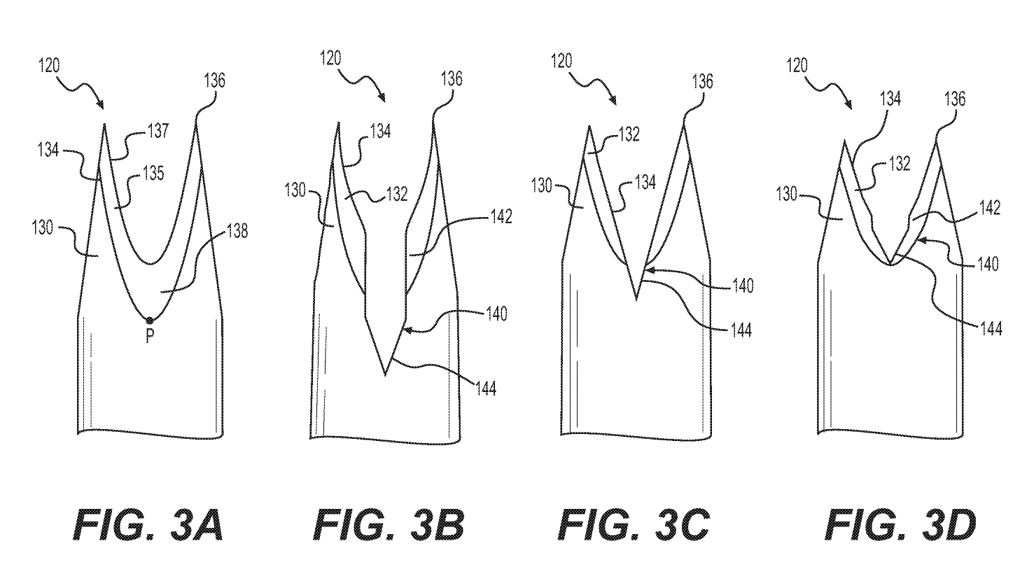

FIG. 3A is a partial front view of distal end 120 of biopsy needle 100 without cutouts 140. FIGS. 3B-3D are partial front views of distal end 120 of biopsy needle 100 having cutouts 140. Exemplary configurations, functions, and/or advantages of cutouts 140 are described below with reference to FIGS. 3A-3D,

As shown in FIG. 3A, without cutouts 140, distal end 120 includes a plurality of tines 130 and a plurality of primary ground bevels 135 formed on different grind planes. Tines 130 and/or the grind planes may be radially evenly spaced apart. Each primary ground bevel 135 is shared by two tines 130, and each tine 130 has a tine tip 136 where two grind planes forming the tine meet. Each ground bevel 135 includes a cutting edge 137 and a bevel heel 138. As shown in FIG. 3A, bevel heel 138 resides within a lowest point “P” of the grind plane of the corresponding ground bevel 135 along the longitudinal axis of biopsy needle 100.

The inclination angle of cutting edge 137 of FIG. 3A is at its highest value at tine tip 136 and decreases towards the bottom of ground bevel 135. The inclination angle is close to or about zero at bevel heel 138 of ground bevel 135. Locations along cutting edge 137 with lower inclination angles, such as bevel heel 138, result in higher cutting forces applied to the tissue during needle insertion, which in turn causes the tissue to be pushed away or around the needle rather than being cut and entering the lumen of the needle. Therefore, distal end 120 of FIG. 3A has poor cutting performance at and near bevel heels 138 of its ground bevels 135.

According to exemplary embodiments of the present disclosure, to improve the cutting performance of biopsy needle 100 of FIG. 3A, distal end 120 of biopsy needle 100 includes a plurality of cutouts 140 residing at locations of ground bevels 135 that have low inclination angles, as shown in FIGS. 3B-3D. For example, cutouts 140 may reside at the locations of bevel heels 138 of ground bevels 135 of FIG. 3A. As shown in FIGS. 3B-3D, cutouts 140 may each cut into or cut through a bevel heel 138, and thus separate each ground bevel 135 into two secondary ground bevels 132, thereby eliminating bevel heels 138 in distal end 120. Each ground bevel 132 then has a separate cutting edge 134. Advantageously, cutouts 140 eliminate or replace locations of cutting edges 137 of distal end 120 having low inclination angles, thereby increasing the overall average inclination angle of distal end 120 and thus the cutting performance of biopsy needle 100. For example, an overall average inclination angle of distal end 120 may range from about 50° to about 85°.

Cutouts 140 can be formed by any suitable micro-machining operation or method, including laser cutting, electrical discharge machining (EDM) cutting, and chemical etching methods. Cutouts 140 can have different configurations. In some exemplary embodiments, as shown in FIG. 3B, each cutout 140 may include a longitudinally straight section 142 and a V-shaped section 144, both of which cut through or extend beyond bevel heel 138 of ground bevel 135. In such instances, both longitudinally straight section 142 and V-shaped section 144 extend beyond point “P” along the longitudinal axis of biopsy needle 100. As described herein, point “P” is the lowest point of the grind plane of ground bevel 132 or the lowest point of the grind plane of ground bevel 135 as shown in FIG. 3A along the longitudinal axis of biopsy needle 100.

The inclination angle of longitudinally straight section 142 is about 90°, greater than the inclination angles of the locations along cutting edge 137 replaced by longitudinally straight section 142. Additionally, the inclination angle of V-shaped section 144 is greater than the inclination angles of locations at or around bevel heel 138 replaced by V-shaped section 144. Therefore, by eliminating or replacing the locations of distal end 120 having low inclination angles, cutouts 140 advantageously increase the overall average inclination angle of distal end 120, thereby reducing cutting forces applied to the tissue and improving the cutting performance of biopsy needle 100.

In other exemplary embodiments, as shown in FIG. 3C, each cutout 140 may only include V-shaped section 144, which cuts through or extends beyond bevel heel 138 of ground bevel 135. In such instances, V-shaped section 144 extends beyond point “P” along the longitudinal axis of biopsy needle 100. Alternatively, as shown in FIG. 3D, cutout 140 may include longitudinally straight section 142 and V-shaped section 144, both of which cut into or reside within bevel heel 138 of ground bevel 135. In such instances, both longitudinally straight section 142 and/or V-shaped section 144 of cutout 140 reside within point “P” along the longitudinal axis of biopsy needle 100.

In further exemplary embodiments, longitudinally straight section 142 of cutout 140 may at least partially reside within bevel heel 138 of ground bevel 135 while V-shaped section 144 may extend beyond bevel heel 138 of ground bevel 135 (not shown). Alternatively, longitudinally straight section 142 of cutout 140 may reside within bevel heel 138 of ground bevel 135 while V-shaped section 144 may partially reside within and partially extend beyond bevel heel 138 of ground bevel 135 (not shown).

As described herein, the widths and/or lengths of longitudinally straight section 142 and V-shaped section 144 of cutout 140 may be predetermined such that locations along ground bevel 135 are eliminated, including bevel heel 138. In some embodiments, biopsy needle 100 may have a hypodermic gauge ranging from about 27 G to about 17 G, or an outer circumference ranging from about 1.294 mm to about 4.628 mm, respectively. In such instances, the sum arc lengths of cutouts 140 are less than about 75% and more than about 10% of the total outer circumference of biopsy needle 100. As described herein, an arc length of cutout 140 is the length of cutout 140 extending along the outer circumference of biopsy needle 100. Increasing the total arc lengths of cutouts 140 increases the overall average inclination angle of biopsy needle 100 and reduces the cutting forces applied to the tissue by biopsy needle 100.

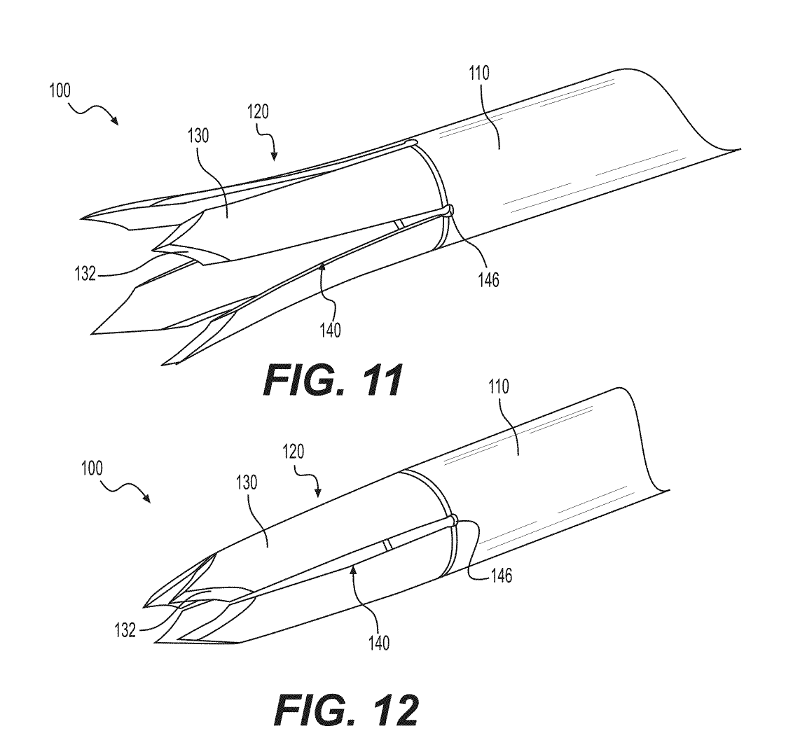

For an exemplary biopsy needle 100 having a hypodermic gauge ranging from about 17 G to about 27 G, the longitudinal lengths of cutouts 140 along the length of biopsy needle 110 may extend to the proximal edge of bevel heel 138 (or point “P” as shown in FIG. 3A) at the minimum. Alternatively, the longitudinal lengths of cutouts 140 may extend beyond the proximal edge of bevel heel 138 (or point “P” as shown in FIG. 3A). In such instances, the longitudinal lengths of cutouts 140 are less than about 15 mm, for example. Increasing the longitudinal lengths of cutouts 140 may increase a degree of radial deflection of tines 130 during needle insertion and/or tissue collection as described below with reference to FIGS. 11 and 12.

Additionally, the widths and/or lengths of longitudinally straight section 142 and V-shaped section 144 of cutout 140 may be predetermined such that the integrity of tines 130 can be maintained during the insertion and/or extraction of biopsy needle 100. As described herein, the integrity of tines 130 may be maintained when tines 130 are wide enough to have sufficient strength to avoid from being bent, deformed, or damaged due to frictional and/or reactive forces from the tissue applied to tines 130 during needle insertion or extraction. Such bending, deformation, or damage of tines 130 may further impede the tissue from entering the lumen of biopsy needle 100 for collection, and/or may result in inadvertent damage to the tissue at the sampling location. Therefore, the widths and/or lengths of cutouts 140 may be predetermined based on the widths of tines 130 and/or the size of ground bevel 135.

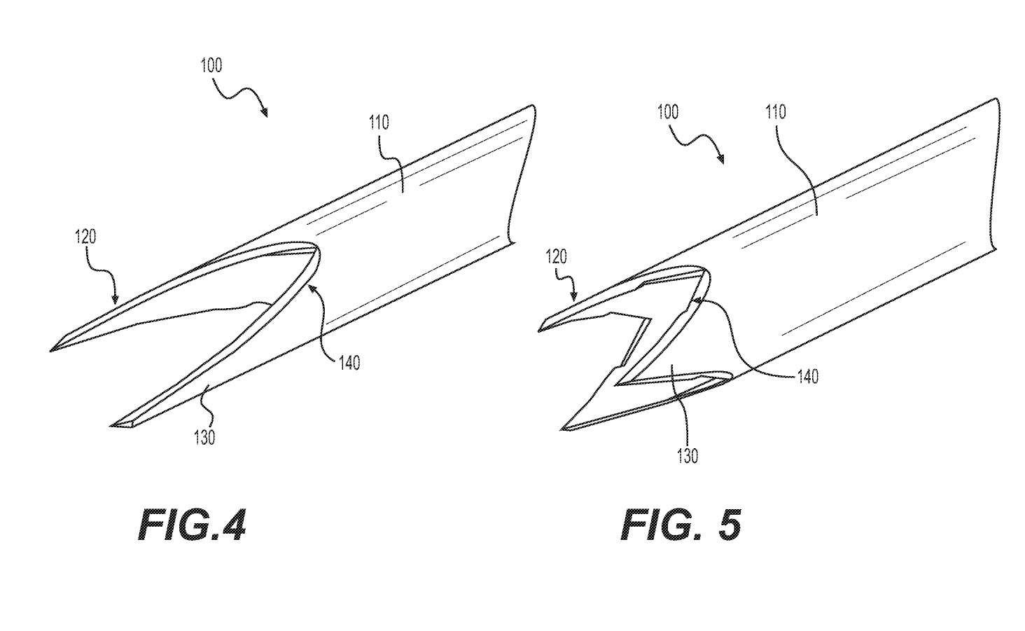

Biopsy needle 100 may have a predetermined number of tines 130 formed on a corresponding number of grind planes suitable for a desired endoscopic biopsy procedure. For example, biopsy needle 100 may include two tines 130 formed by two grind planes as shown in FIG. 4 (two-plane biopsy needle 100) or three tines 130 formed by three grind planes as shown in FIG. 5 (three-plane biopsy needle 100). In such instances, distal end 120 of biopsy needle 100 includes a same number of cutouts 140 located between each set of adjacent tines 130. In some embodiments, distal end 120 of biopsy needle 100 includes four tines 130 formed by four grind planes (four-plane biopsy needle 100). Four tines 130 of a four-plane biopsy needle 100 are evenly radially spaced apart in rotational symmetry as well as in plane symmetry about two orthogonal longitudinal planes that are parallel to the longitudinal axis of biopsy needle 100.

For example, as shown in FIGS. 1 and 2, distal end 120 includes four tines 130 formed by four grind planes. Distal end 120 further includes eight ground bevels 132 and eight cutting edges 134. Each of the grind planes is oblique to the longitudinal axis of biopsy needle 100 and orthogonal to two neighboring grind planes in an x-y plane perpendicular to the longitudinal axis. Each of the four grind planes is oblique to the longitudinal axis at a desired bevel angle ranging from about 5° to about 20°. In some embodiments, the four grind planes are oblique to the longitudinal axis at the same desired bevel angle.

As described herein, the bevel angle of the grind planes of biopsy needle 100 may be selected based on various factors, such as the hardness or softness of the tissue to be sampled, the gauge of biopsy needle 100, the number of tines 130, the geometry of tines 130, etc. Decreasing the bevel angles of the grind planes increases the inclination angles of cutting edges 134, and thus increases the overall average inclination angle of biopsy needle 100. This in turn reduces the cutting forces applied to the tissue by distal end 120 of biopsy needle 100, allowing for more efficient tissue cutting and better tissue samples for biopsy.

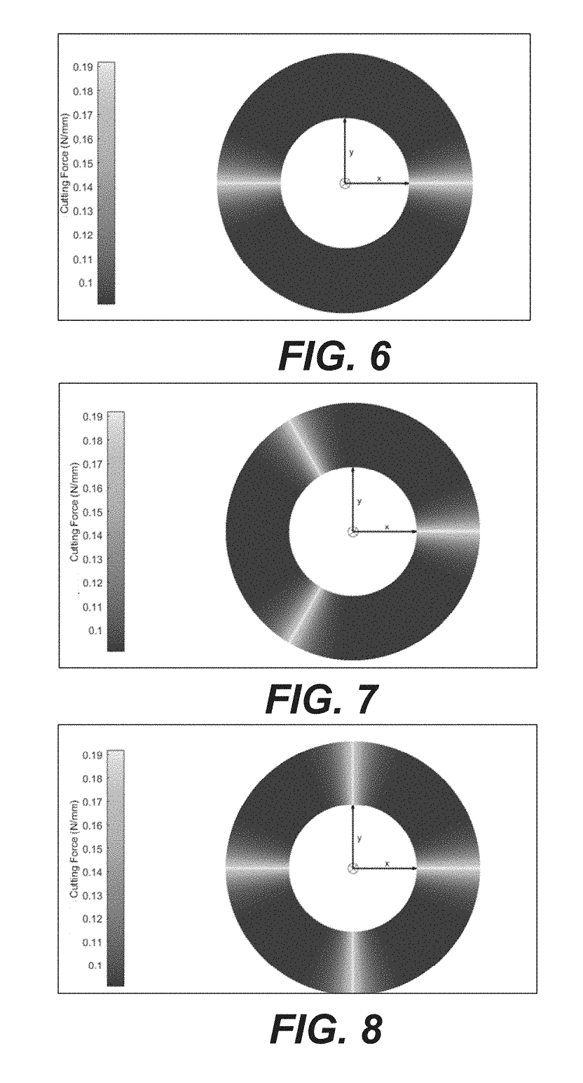

Advantageously, compared to biopsy needles 100 having two tines 130 or three tines 130, biopsy needle 100 having four or a higher, even number of tines 130 increases the success rate of collecting adequate tissue samples as described below with reference to FIGS. 6-8.

FIG. 6 is a graphical illustration for an exemplary distribution of cutting force along distal end 120 of two-plane biopsy needle 100 of FIG. 4. FIG. 7 is a graphical illustration for an exemplary distribution of cutting force along distal end 120 of three-plane biopsy needle 100 of FIG. 5. FIG. 8 is a graphical illustration for an exemplary distribution of cutting force along distal end 120 of the four-plane biopsy needle 100 of FIG. 1.

As shown in FIGS. 6-8, during needle insertion, cutting force applied to the tissue being cut is at the lowest at tine tip 136 and increases with decreasing inclination angle along cutting edge 134 or cutting edge 137. Two-plane biopsy needle 100 of FIG. 4 is symmetric over a single longitudinal plane but not over the orthogonal longitudinal plane. Thus, as shown in FIG. 6, cutting force applied to the tissue being cut by two-plane biopsy needle 100 is only balanced about one longitudinal plane of biopsy needle 100. Three-plane biopsy needle 100 of FIG. 5 is rotationally symmetric but it not symmetric over a longitudinal plane. Thus, as shown in FIG. 7, cutting force applied to the tissue being cut of three-plane biopsy needle 100 is only rotationally symmetric, not balanced about any longitudinal plane of biopsy needle 100. The lack of balance of cutting force along two longitudinal planes allow the tissue being cut to be pushed away or around biopsy needle 100, resulting in loss of tissue cut by distal end 120.

As described herein, one or more parameters of biopsy needle 100 may affect the amount of cutting force applied to the tissue being cut by distal end 120, such as the material, gauge, thickness, number of tines of biopsy needle 100. The values of cutting force of biopsy needle 100 as shown in FIGS. 6-8 are exemplary.

Advantageously, four-plane biopsy needle 100 of FIGS. 1 and 2 has both rotational symmetry and plane symmetry about two orthogonal longitudinal planes of biopsy needle 100. The combination of both rotational and plane symmetry allows substantially balanced cutting force to be applied to the tissue by distal end 120 of biopsy needle 100 during needle insertion. The substantially balanced cutting force allows the tissue being cut to move towards the center of biopsy needle 100 and to enter the lumen of the biopsy needle 100. Otherwise, the tissue would have been pushed away from or pushed around the needle by unbalanced cutting force, as for the two-plane and three-plane biopsy needles. Thus, the rotational symmetric and plane symmetric arrangement of four-plane biopsy needle 100 of FIGS. 1 and 2 advantageously reduces loss of tissue and/or increases the amount and/or length of the collected tissue sample, thereby increasing the success rate of collecting an adequate tissue sample. The rotational symmetric and plane symmetric arrangement of four-plane biopsy needle 100 of FIGS. 1 and 2 may further reduce the inadvertent damage to the surrounding tissue at the sampling location.

Additionally, the substantially balanced cutting force applied to the tissue being cut in turn results in substantially balanced reactive force of the tissue applied to distal end 120. The substantially balanced reactive force prevents a four-plane biopsy needle 100 from being veered off course, changing direction, and/or curving in undesired directions during tissue sample collection. Thus, the rotational symmetric and/or plane symmetric arrangement of a four-plane biopsy needle 100 of FIGS. 1 and 2 advantageously allows the biopsy needle to maintain a straight sampling path, thereby increasing the accuracy of sampling a desired legion location.

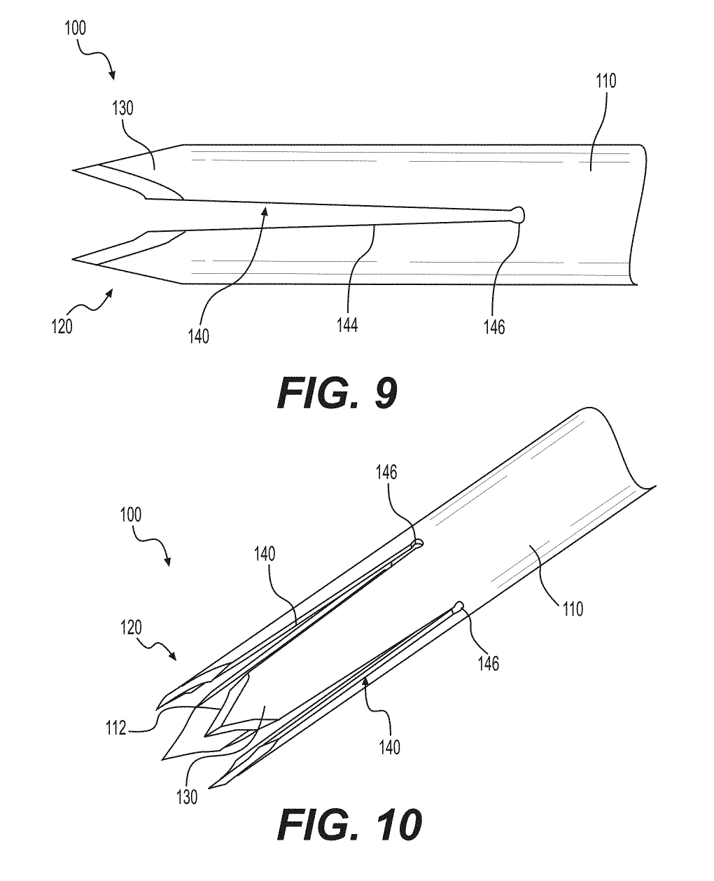

FIG. 9 is a partial front view of another exemplary biopsy needle 100 whose cutouts 140 along the longitudinal axis of biopsy needle 100 are longer than the widths of tines 130. FIG. 10 is a partial perspective view of the exemplary biopsy needle 100 of FIG. 9.

As shown in FIGS. 9 and 10, cutouts 140 may include a V-shaped section 144, for example. V-shaped section 144 may be substantially longer than the widths of tines 130, thereby allowing the tines to be radially deflectable. Cutout 140 may further include a strain relief section 146, such as an opening, at the proximal edge of V-shaped section 144. Strain relief section 146 reduces the risk of tines 130 of being bent, deformed, damaged, or dislodged from biopsy needle 100.

As shown in FIG. 11, when biopsy needle 100 is inserted into a sampling location, the longer cutouts 140 residing between tines 130 may allow tines 130 to radially deflect outward relatively to the longitudinal axis of biopsy needle 100. Such deflection may be enabled by the reactive force of the tissue applied to tines 130. This outward radial deflection advantageously allows more tissue to enter the lumen of biopsy needle 100 of a given gauge more easily (e.g., with less hindrance), thereby allowing for collecting a greater amount and/or length of tissue sample.

Additionally, as shown in FIG. 12, when biopsy needle 100 is extracted from a sampling location, the longer cutouts 140 residing between tines 130 may allow tines 130 to radially collapse or deflect inward relatively to the longitudinal axis of biopsy needle 100. This inward radial deflection of tines 130 advantageously compresses the tissue sample but by distal end 120, and prevents the cut tissue sample from exiting the lumen of biopsy needle 100 (e.g., by more frictional and/or supporting surface), thereby preventing loss of tissue cut by distal end 120 and increasing the success rate of collecting adequate tissue sample.

As described herein, the lengths and widths of cutouts 140 may be selected based on a suitable degree of radial expansion and collapse of tines 130 for a desired endoscopic procedure. Longer and/or wider cutouts 140 may increase the degree of radial expansion of tines 130 during needle insertion. Also, longer or wider cutouts 140 may increase the degree of radial collapse of tines 130, producing a more pronounced grabbing or pulling motion on the cut tissue during needle extraction. The degree of radial expansion and/or collapse of tines 130 may be predetermined as needed for the desired endoscopic tissue sampling procedure.

The foregoing description has been presented for purposes of illustration. It is not exhaustive and is not limited to precise forms or embodiments disclosed. Modifications and adaptations of the embodiments will be apparent from consideration of the specification and practice of the disclosed embodiments. In addition, while certain components have been described as being coupled to one another, such components may be integrated with one another or distributed in any suitable fashion.

Moreover, while illustrative embodiments have been described herein, the scope includes any and all embodiments having equivalent elements, modifications, omissions, combinations (e.g., of aspects across various embodiments), adaptations and/or alterations based on the present disclosure. The elements in the claims are to be interpreted broadly based on the language employed in the claims and not limited to examples described in the present specification or during the prosecution of the application, which examples are to be construed as nonexclusive.

The features and advantages of the disclosure are apparent from the detailed specification, and thus, it is intended that the appended claims cover all systems and methods falling within the true spirit and scope of the disclosure. As used herein, the indefinite articles “a” and “an” mean “one or more.” Similarly, the use of a plural term does not necessarily denote a plurality unless it is unambiguous in the given context. Words such as “and” or “or” mean “and/or” unless specifically directed otherwise. Further, since numerous modifications and variations will readily occur from studying the present disclosure, it is not desired to limit the disclosure to the exact construction and operation illustrated and described, and accordingly, all suitable modifications and equivalents may be resorted to, falling within the scope of the disclosure.

Other embodiments will be apparent from consideration of the specification and practice of the embodiments disclosed herein. It is intended that the specification and examples be considered as example only, with a true scope and spirit of the disclosed embodiments being indicated by the following claims.

What is claimed is:

1. A biopsy needle, comprising:
an elongated body extending along a longitudinal axis, the elongated body comprising a lumen extending therethrough and a distal end;
the distal end comprising
at least four tines, each tine comprising two ground bevels formed on two grind planes; and
a plurality of cutouts, each cutout residing between two adjacent tines and comprising a V-shaped section.

2. The biopsy needle of claim 1, wherein the tines are evenly radially spaced apart in rotational symmetry.

3. The biopsy needle of claim 1, wherein the tines are arranged in plane symmetry about two longitudinal planes being parallel to the longitudinal axis and orthogonal to each other.

4. The biopsy needle of claim 1, wherein each of the two grind planes is oblique relative to the longitudinal axis at a bevel angle ranging from 5° to 20°.

5. The biopsy needle of claim 1, wherein the two grind planes of each of the tines are oblique relative to the longitudinal axis at a same bevel angle.

6. The biopsy needle of claim 1, wherein the distal end comprises four tines evenly radially spaced apart in rotational symmetry.

7. The biopsy needle of claim 1, wherein each of the ground bevels comprises a cutting edge.

8. The biopsy needle of claim 1, wherein an overall average inclination angle of the distal end ranges from 50° to 85°.

9. The biopsy needle of claim 1, wherein each of the cutouts further comprises a longitudinally straight section.

10. The biopsy needle of claim 9, wherein the V-shaped section and/or longitudinally straight section reside within a lowest point of the grind planes along the longitudinal axis.

11. The biopsy needle of claim 9, wherein the V-shaped section and/or the longitudinally straight section extend beyond a lowest point of the grind planes along the longitudinal axis.

12. The biopsy needle of claim 1, wherein the lengths of the cutouts along the longitudinal axis are smaller than or equal to the widths of the tines.

13. The biopsy needle of claim 1, wherein the widths of the cutouts are smaller than or equal to the widths of the tines.

14. The biopsy needle of claim 1, wherein the sum of the arc lengths of the cutouts are more than 10% and less than 75% of the outer circumference of the biopsy needle.

15. The biopsy needle of claim 1, wherein the lengths of the cutouts along the longitudinal axis are greater than the widths of the tines.

16. The biopsy needle of claim 15, wherein the tines are capable of radially deflecting outward relatively to the longitudinal axis as the biopsy needle penetrates a sample tissue and radially collapsing inward as the biopsy needle extracts from the sample tissue.

17. The biopsy needle of claim 1, wherein the cutout is formed by a method selected from a group of micro-machining operations, including laser cutting, electrical discharge machining (EDM) cutting, and chemical etching.

18. A device for needle biopsy, the device comprising:
a biopsy needle, the biopsy needle comprising an elongated body extending along a longitudinal axis and a distal end, the distal end comprising
at least three tines, each tine comprising two ground bevels formed on two grind planes; and
a plurality of cutouts, each cutout residing between two adjacent tines and comprising a V-shaped section.

19. The biopsy needle of claim 18, wherein each of the cutouts further comprises a longitudinally straight section.

20. A biopsy needle tip, comprising:
at least two tines, each tine comprising two ground bevels formed on two grind planes; and
a plurality of cutouts, each cutout residing between two adjacent tines and comprising a V-shaped section; wherein the lengths of the cutouts along the longitudinal axis are greater than the widths of the tines.

21. The biopsy needle of claim 20, wherein each of the cutouts further comprises a longitudinally straight section.
